(19) United States
(12) Patent Application Publication
(10) Pub. No.: US 2011/0218324 A1
(43) Pub. Date: Sep. 8, 2011

(54) PREPARING POLYETHER ALCOHOLS

(75) Inventors: Sirus ZARBAKHSH, Central Hong Kong (CN); Markus SCHÜTTE, Osnabrueck (DE); Holger SEIFERT, Bohmte (DE); Vinit CHILEKAR, Mannheim (DE); Berend ELING, Lemfoerde (DE); Jan RUDLOFF, Ann Arbor, MI (US)

(73) Assignee: BASF SE, Ludwigshafen (DE)

(21) Appl. No.: 13/037,504

(22) Filed: Mar. 1, 2011

Related U.S. Application Data
(60) Provisional application No. 61/309,471, filed on Mar. 2, 2010.

(51) Int. Cl. C08G 65/34; C07C 41/03; C07C 43/13

(52) U.S. Cl. 528/425; 568/620; 568/622

(57) ABSTRACT

In a process for preparing a polyether alcohol d1) by reaction of a) at least one compound having at least three hydrogen atoms reactive with alkylene oxides and a molecular weight Mn of not more than 600 g/mol with b) an alkylene oxide by using c) a catalyst, said reaction is carried out in the presence of a polyether alcohol d) having a hydroxyl number of 100-800 mg KOH/g and a functionality of 1.5 to 8.

The present invention relates to a process for preparing polyether alcohols by addition of alkylene oxides onto compounds having hydrogen atoms reactive with alkylene oxides.

Preparing polyether alcohols by addition of alkylene oxides onto compounds having reactive hydrogen atoms, hereinafter also referred to as starter compounds, is long known. The addition is usually carried out using a catalyst.

Polyether alcohols used in the manufacture of rigid polyurethane foams usually have a functionality in the range from 2 to 8 and a hydroxyl number in the range between 250 and 800 mgKOH/g. Compounds used having at least two hydrogen atoms reactive with alkylene oxides are usually alcohols and/or amines having 2 or more reactive hydrogen atoms. They can be polyfunctional alcohols, such as glycerol, diethylene glycol, trimethylolpropane, sorbitol or sucrose, or amines, such as ethylenediamine (EDA), toluenediamine (TDA) or diphenylmethanediamine (MDA) or polymeric MDA (p-MDA). Particularly in the case of solid starters, only slow metering rates are possible at the beginning of the operation, since the alkylene oxide is only sparingly soluble in the reaction mixture. The result is an alkylene oxide accumulation which manifests as a high internal reactor pressure. The high accumulation of alkylene oxide represents an unsafe state, since a runaway reaction would result in exceedance of the reactor's design pressure. Furthermore, the high viscosity results in poor removal of heat. This leads to long cycle times. Moreover, the poor removal of heat can lead to local overheating—hot spots—which have an adverse effect on product quality. High viscosity causes the pumps and the external heat exchanger to wear at an accelerated rate.

WO 99/47581 describes a process for preparing polyether alcohols using sugars and co-initiators which utilizes imidazoles as catalysts. The starter mixture may also comprise a polyether alcohol. The alkylene oxides are metered into the reaction at a slow rate, leading to a low space-time yield.

It is an object of the present invention to develop a process that makes it possible to prepare polyether alcohols with an improved space-time yield while avoiding the disadvantages of the prior art. The process should be simple and should also allow the use of amines as catalysts.

We have found that this object is achieved, surprisingly, when the starter mixture comprises at least one compound having at least three hydrogen atoms reactive with alkylene oxides and a molecular weight Mn of not more than 600 g/mol and preferably not more than 400 g/mol, and at least one polyether alcohol having a hydroxyl number of 100-800 mg KOH/g and a functionality of 1.5 to 8, and when the stirring energy input is preferably in the range from 0.15 kW/m³ to 5 kW/m³. What is referred to here as the starter mixture is the mixture of the starter compounds and catalyst.

The present invention accordingly provides a process for preparing a polyether alcohol d1) by reaction of a) compounds having at least three hydrogen atoms reactive with alkylene oxides and a molecular weight Mn of not more than 600 g/mol and preferably not more than 400 g/mol with b) an alkylene oxide by using c) a catalyst, wherein said reaction is carried out in the presence of a polyether alcohol d) having a hydroxyl number of 100-800 mg KOH/g and a functionality of 1.5 to 8.

The present invention further provides the polyether alcohols obtained by the process of the present invention and also a method of using them in the manufacture of polyurethanes.

The amount in which the polyether alcohol d) is used is preferably 1-70% by weight, more preferably 10-40% by weight and even more preferably 15-30% by weight, all based on the sum total of compounds a) and d).

In a particularly preferred embodiment of the process of the present invention, said polyether alcohol d) is prepared using the same alkylene oxide b) and the same compound having at least three hydrogen atoms reactive with alkylene oxides a) both in the same amount as in the preparation of said polyether alcohol d1). In other words, the polyether alcohols d) and d1) are the same in this case.

Component a) preferably comprises alcohols having at least two and preferably at least three hydroxyl groups, or amines having at least one amino group. Preference is given to using trimethylopropane, glycerol, pentaerythritol, sugar compounds such as for example glucose, sorbitol, mannitol and sucrose, polyhydric phenols, resols, for example oligomeric condensation products of phenol and formaldehyde and Mannich condensates of phenols, formaldehyde and dialkanolamines, and also melamine and also mixtures of at least two of the alcohols listed.

In a preferred embodiment of the invention, component a) comprises at least one hydroxyl-containing compound that is solid at room temperature. Mannich condensates are a possibility in one embodiment. In a further embodiment, sugar alcohols are concerned. These are preferably selected from the groups comprising glucose, sorbitol, mannitol and sucrose and more preferably sorbitol and sucrose. Sucrose is concerned in a further embodiment.

In a further preferred embodiment of the invention, component a) comprises a mixture of at least one room temperature solid alcohol and at least one room temperature liquid alcohol. The room temperature solid alcohols preferably comprise the sugar alcohols more particularly characterized above, especially sucrose. The room temperature liquid alcohols preferably comprise ethylene glycol and its higher homologs, propylene glycol and its higher homologs and glycerol, especially glycerol. Component a) may also comprise water. When water is used, its amount is more particularly not more than 10% by weight, based on the weight of component a).

A further preferred embodiment of the invention comprises using amines as component a). Aliphatic amines may be concerned. Aromatic amines are preferred. Particular preference is given to using amines having at least one amino group and preferably having at least two amino groups. More particularly, the aromatic amines are selected from the group comprising toluenediamine (TDA) or diphenylmethanediamine (MDA) or polymeric MDA (p-MDA). In the case of TDA it is more particularly the 2,3- and 3,4-isomers, also known as vicinal TDA, which are used.

Alkylene oxide b) preferably comprises propylene oxide, ethylene oxide, butylene oxide, isobutylene oxide, styrene oxide and mixtures of two or more thereof. Preferably, propylene oxide, ethylene oxide or mixtures of propylene oxide and ethylene oxide are used as alkylene oxide b). It is particularly preferable to use propylene oxide as alkylene oxide b).

The catalysts used are usually basic compounds. They are usually the hydroxides of alkali metals, more particularly potassium hydroxide, in commercial scale processes. Since these catalysts disrupt the further processing of the polyether alcohols, they have to be removed from the polyether alcohol, usually by neutralization with an acid and subsequent removal of the resulting salts. This additional operation leads to a reduced space-time yield and to product losses.

This disadvantage can be remedied by using catalysts that can remain in the product. It is accordingly known to use amines as catalysts. Since amines are catalytically less active than the alkaline catalysts, they are preferably used for preparing polyether alcohols having short polyether chains, as preferentially used in the manufacture of rigid polyurethane foams. Polyols of this type are described for example in RO 108246 and R085851 or EP0318784.

It is also possible to use a combination of alkali metal hydroxide and amine catalysts. This is particularly an option to prepare polyols of low hydroxyl number. The products obtained can be worked up similarly to the polyols catalyzed with alkali metal hydroxide. Alternatively, they can also be worked up by performing just the neutralization step with an acid. In this case, it is preferable to use carboxylic acids such as for example lactic acid, acetic acid or 2-ethylhexanoic acid.

It is therefore preferable to use an amine as catalyst c). The amine may be a primary, secondary or tertiary amine. It may further be an aliphatic or aromatic amine. Among aliphatic amines, tertiary amines are particularly preferred. Amino alcohols also count as amines. In one embodiment of the process of the present invention, the amines may comprise aromatic heterocyclic compounds having at least one and preferably at least two nitrogen atoms in the ring.

The amines are preferably selected from the group comprising trialkylamines, more particularly trimethylamine, triethylamine, tripropylamine, tributylamine, dimethylalkylamines, more particularly dimethylethanolamine, dimethylcyclohexylamine, dimethylethylamine, dimethylbutylamine, aromatic amines, more particularly dimethylaniline, dimethylaminopyridine, dimethylbenzylamine, pyridine, imidazoles (more particularly imidazole, N-methylimidazole, 2-methylimidazole, 4-methylimidazole, 5-methylimidazole, 2-ethyl-4-methylimidazole, 2,4-dimethylimidazole, 1-hydroxypropylimidazole, 2,4,5-trimethylimidazole, 2-ethylimidazole, 2-ethyl-4-methylimidazole, N-phenylimidazole, 2-phenylimidazole, 4-phenylimidazole), guanidine, alkylated guanidines (more particularly 1,1,3,3-tetramethylguanidine), 7-methyl-1,5,7-triazabicyclo[4.4.0]dec-5-ene, amidines (more particularly 1,5-diazobicyclo[4.3.0]non-5-ene, 1,5-diazabicyclo[5.4.0]undec-7-ene).

Aminic catalysts can be used alone or mixed with each other in any proportion.

Dimethylethanolamine is catalyst c) in a preferred embodiment of the invention.

An imidazole is catalyst c) in a preferred embodiment of the invention.

The amine is preferably used therein in an amount of 0.1-1.0% by mass based on the overall batch. This amount is particularly preferred when aliphatic amines are used.

Heterocyclic compounds, more particularly imidazoles, are preferably used in an amount of 0.01% to 0.5% by mass based on the overall batch.

A further embodiment of the invention comprises using, as described, oxides, hydroxides or alkoxides of an alkali or alkaline earth metal as catalyst c). The catalyst is more preferably selected from the group comprising potassium hydroxide, cesium hydroxide or potassium tert-butoxide.

To carry out the process of the present invention, the constituents of the starter mixture are mixed together. Next it is preferable to replace initially the gas phase in the reactor by an inert gas, more particularly nitrogen. Next the alkylene oxide is metered.

The addition reaction of the alkylene oxides is preferably carried out at a temperature between 90 and 150° C. and a pressure between 0.1 to 6 bar. The metering of the alkylene oxides is typically followed by a postreaction phase to complete the reaction of the alkylene oxides.

It is preferable for the first 40% by weight more preferably the first 30% by weight and especially 25% by weight of the amounts of alkylene oxide needed to be at an average metering rate of 45 kg/h/m³ to 160 kg/h/m³, more preferably 50 kg/h/m³ to 140 kg/h/m³, and especially 75 kg/h/m³ to 120 kg/h/m³. The stated volume relates to the reactor volume. Any external cooling circuit is included in the reactor volume.

The average metering rate based on the reactor volume is computed by the alkylene oxide quantity added being divided by the metering time needed for this alkylene oxide quantity and by the reactor volume.

The process of the present invention is advantageously conducted with a stirring energy input of 0.15 kW/m³ to 4.5 kW/m³, preferably 0.25 kW/m³ to 2.8 kW/m³ and more preferably 0.3 kW/m³ to 2.0 kW/m³.

Conclusion of the metering of the alkylene oxides is typically followed by a postreaction phase in which the reaction of the alkylene oxide is taken to completion. This is typically followed by a workup of the reaction product, for example by a distillation to remove volatiles, which is preferably carried out under reduced pressure. It is further possible to strip with inert gas or steam during, before or after the distillation. Stripping takes place in a temperature range of 60-150° C. and a pressure range of 15-1013 mbar. The inert gas or steam is introduced at 1-1900 kg/h/m³. The stated volume is based on the reactor volume.

When oxides, hydroxides or alkoxides of an alkali or alkaline earth metal are used, the reaction mixture following the addition reaction of the alkylene oxides is dewatered by distillation and also worked up by acid neutralization and removal of the salts formed.

As mentioned, the polyether alcohols of the present invention can be reacted with polyisocyanates to form rigid polyurethane foams.

The starting materials used for this may be more particularly described as follows:

The organic polyisocyanates contemplated are preferably aromatic polyfunctional isocyanates.

Specific examples are: 2,4- and 2,6-tolylene diisocyanate (TDI) and the corresponding isomeric mixtures, 4,4′-, 2,4′- and 2,2′-diphenylmethane diisocyanate (MDI) and the corresponding isomeric mixtures, mixtures of 4,4′- and 2,4′-diphenylmethane diisocyanates and in the manufacture of rigid polyurethane foams particularly mixtures of 4,4′-, 2,4′- and 2,2′-diphenylmethane diisocyanates and polyphenyl polymethylene polyisocyanates (crude MDI).

The polyether alcohols of the present invention are typically used in admixture with other compounds having at least two hydrogen atoms reactive with isocyanate groups.

Compounds useful together with the polyether alcohols d1) used according to the present invention and having at least two isocyanate-reactive hydrogen atoms include particularly polyether alcohols and/or polyester alcohols having OH numbers in the range from 100 to 1200 mgKOH/g.

The polyester alcohols used together with the polyether alcohols used according to the present invention are usually prepared by condensation of polyfunctional alcohols, preferably diols, having 2 to 12 carbon atoms and preferably 2 to 6 carbon atoms, with polyfunctional carboxylic acids having 2 to 12 carbon atoms, for example succinic acid, glutaric acid, adipic acid, suberic acid, azelaic acid, sebacic acid, decanedicarboxylic acid, maleic acid, fumaric acid and preferably phthalic acid, isophthalic acid, terephthalic acid and the isomeric naphthalenedicarboxylic acids.

The polyether alcohols used together with the polyether alcohols d1) used according to the present invention usually have a functionality between 2 and 8 and more particularly from 3 to 8.

Particular preference is given to using polyether alcohols prepared by known methods, for example by anionic polymerization of alkylene oxides in the presence of catalysts.

The compounds having at least two isocyanate-reactive hydrogen atoms also include the optionally used chain extenders and crosslinkers. Rigid polyurethane films can be manufactured with or without the use of chain-extending and/or crosslinking agents. The addition of difunctional chain-extending agents, trifunctional and higher-functional crosslinking agents or optionally also mixtures thereof may prove advantageous for modifying the mechanical properties. Chain-extending and/or crosslinking agents used are preferably alkanolamines and, more particularly, diols and/or triols having molecular weights of below 400, preferably in the range from 60 to 300.

Chain-extending agents, crosslinking agents or mixtures thereof are advantageously used in an amount of 1% to 20% by weight and preferably 2% to 5% by weight, based on the polyol component.

The polyurethane foams are typically manufactured in the presence of a blowing agent. The blowing agent used may preferably be water, which reacts with isocyanate groups by elimination of carbon dioxide. So-called physical blowing agents can also be used in addition to or in lieu of water. Physical blowing agents comprise usually room temperature liquid compounds which are inert toward the feed components and vaporize under the conditions of the urethane reaction. The boiling point of these compounds is preferably below 50° C. Physical blowing agents also include compounds which are gaseous at room temperature and are introduced into and/or dissolved in the feed components under pressure, examples being carbon dioxide, low-boiling alkanes and fluoroalkanes.

Physical blowing agents are usually selected from the group comprising alkanes and/or cycloalkanes having at least 4 carbon atoms, dialkyl ethers, esters, ketones, acetals, fluoroalkanes having 1 to 8 carbon atoms and tetraalkylsilanes having 1 to 3 carbon atoms in the alkyl chain, more particularly tetramethylsilane.

Manufacturing polyurethanes may be effected, if necessary, in the presence of catalysts, flame retardants and also customary auxiliary and/or added substances.

Further particulars concerning the starting compounds used may be found for example in Kunststoffhandbuch, volume 7 “Polyurethane”, edited by Gunter Oertel, Carl-Hanser-Verlag Munich, 3rd edition, 1993.

The examples which follow illustrate the invention.

### EXAMPLE 1

### Polyetherol 1

A 300 ml pressure reactor was charged with 21.15 g of glycerol, 58.68 g of sucrose and 1.76 g of dimethylethanolamine. The stirrer was started (0.1 kW/m³) and the reactor was repeatedly inertized and heated to 105° C. Next the metering of propylene oxide was commenced at a rate of 0.8 ml/minute. Before the quantity of propylene oxide metered reached 35 g, the reactor pressure exceeded 6.4 bar, and the run had to be discontinued for safety reasons. The desired product could not be isolated. The target product was a polyether alcohol having a hydroxyl number of 500 mgKOH/g.

### EXAMPLE 2

### Polyetherol 2

The reactor described in Example 1 was charged with 18.02 g of glycerol, 31.2 g of a polyol of hydroxyl number 500 mgKOH/g and functionality 4.95, 50.2 g of sucrose and 1.52 g of dimethylethanolamine. The stirrer was started (0.1 kW/m³) and the reactor was repeatedly inertized and heated to 105° C. Next the metering of propylene oxide was commenced at a rate of 0.8 ml/minute. Before the quantity of propylene oxide metered reached 35 g, the reactor pressure exceeded 6.4 bar, and the run had to be discontinued for safety reasons. The desired product could not be isolated. The target product was a polyether alcohol having a hydroxyl number of 500 mgKOH/g.

### EXAMPLE 3

### Polyetherol 3

The reactor described in Example 1 was charged with 18.13 g of glycerol, 31.1 g of polyol of hydroxyl number 500 mgKOH/g and functionality 5.95, 50.2 g of sucrose and 1.50 g of dimethylethanolamine. The stirrer was started (0.5 kW/m³) and the reactor was repeatedly inertized and heated to 105° C. Next the metering of propylene oxide was commenced at a rate of 0.8 ml/minute. During the feeding of the first 35 g of propylene oxide (metering time: 53 minutes) the pressure maximum was 5 bar. Altogether 126.5 g of propylene oxide were introduced (total metering time: 190 minutes). The average metering rate based on the reactor volume was 133 kg/h/m³. The postreaction of 3 hours took place at 112° C. The propylene oxide still present was stripped off in a nitrogen stream. The product (220 g) had the following parameters:

| | | |
|---|---|---|
| hydroxyl number | 507 | mg KOH/g |
| viscosity at 25° C. | 17 123 | mPas |

| | |
|---|---|
| water content | 0.009% |
| pH | 10.3 |

Polyol viscosity, unless otherwise stated, was determined at 25° C. using a Rheotec RC 20 rotary viscometer with spindle CC 25 DIN (spindle diameter: 12.5 mm, measuring cylinder internal diameter: 13.56 mm) at a shear rate of 50 1/s.

We claim:

1. A process for preparing a polyether alcohol d1) by reaction of a) at least one compound having at least three hydrogen atoms reactive with alkylene oxides and a molecular weight Mn of not more than 600 g/mol with b) an alkylene oxide by using c) a catalyst, wherein said reaction is carried out in the presence of a polyether alcohol d) having a hydroxyl number of 100-800 mg KOH/g and a functionality of 1.5 to 8.

2. The process according to claim 1 wherein said polyether alcohol d) is used in an amount of 1-70% by weight based on the sum total of said compounds a) and d).

3. The process according to claim 1 wherein alkylene oxide is metered at an average metering rate based on the reactor volume of 45 kg/h/m³ to 160 kg/h/m³.

4. The process according to claim 1 wherein the stirring energy input is in the range from 0.15 kW/m³ to 5 kW/m³.

5. The process according to claim 1 wherein said polyether alcohol d1) is prepared using the same alkylene oxide b) and the same compound having at least three hydrogen atoms reactive with alkylene oxides a) both in the same amount as in the preparation of said polyether alcohol d).

6. The process according to claim 1 wherein said alkylene oxide b) comprises propylene oxide, ethylene oxide, butylene oxide, isobutylene oxide, styrene oxide and mixtures of two or more thereof.

7. The process according to claim 1 wherein component a) comprises alcohols having at least three hydroxyl groups.

8. The process according to claim 1 wherein said component a) comprises at least one sugar alcohol.

9. The process according to claim 1 wherein said component a) is a mixture of at least one room temperature solid alcohol and at least one room temperature liquid alcohol.

10. The process according to claim 1 wherein component a) comprises amines having at least two amino groups.

11. The process according to claim 1 wherein catalyst c) comprises an amine.

12. The process according to claim 1 wherein said catalyst c) is an amine selected from the group comprising trialkylamines, more particularly trimethylamine, triethylamine, tripropylamine, tributylamine, dimethylalkylamines, more particularly dimethylethanolamine, dimethylcyclohexylamine, dimethylethylamine, dimethylbutylamine, aromatic amines, more particularly dimethytaniline, dimethylaminopyridine, dimethylbenzylamine, pyridine, imidazoles (more particularly imidazole, N-methylimidazole, 2-methylimidazole, 4-methylimidazole, 5-methylimidazole, 2-ethyl-4-methylimidazole, 2,4-dimethylimidazole, 1-hydroxypropylimidazole, 2,4,5-trimethylimidazole, 2-ethylimidazole, 2-ethyl-4-methylimidazole, N-phenylimidazole, 2-phenylimidazole, 4-phenylimidazole), guanidine, alkylated guanidines (more particularly 1,1,3,3-tetramethylguanidine), 7-methyl-1,5,7-triazabicyclo[4.4.0]dec-5-ene, amidines (more particularly 1,5-diazobicyclo[4.3.0]non-5-ene, 1,5-diazabicyclo[5.4.0]undec-7-ene).

13. The process according to claim 1 wherein said catalyst c) is dimethylethanolamine. The process according to claim 1 wherein the catalyst is imidazole.

14. The process according to claim 1 wherein said catalyst c) is the oxide, hydroxide or alkoxide of an alkali or alkaline earth metal.

15. A polyether alcohol obtainable according to any one of claims 1 to 14.

16. The use of a polyether alcohol according to claim 15 in the manufacture of polyurethanes.
